(19) United States
(12) Patent Application Publication
(10) Pub. No.: US 2009/0136579 A1
(43) Pub. Date: May 28, 2009

(54) Nanoparticles Comprising a PDGF Receptor Tyrosine Kinase Inhibitor

(76) Inventor: Kensuke Egashira, Fukuoka (JP)

Correspondence Address: WENDEROTH, LIND & PONACK, L.L.P., 1030 15th Street, N.W.,, Suite 400 East, Washington, DC, 20005-1503, US

(21) Appl. No.: 12/225,539

(22) PCT Filed: Mar. 23, 2007
(86) PCT No.: PCT/JP2007/057024

§ 371 Date: Sep. 29, 2008

Related U.S. Application Data
(60) Provisional application No. 60/785,576, filed on Mar. 24, 2006.

(51) Int. Cl. A61K 9/16; A61K 31/506

(52) U.S. Cl. 424/489; 514/252.18; 424/501; 977/773

(57) ABSTRACT

The present invention relates to nanoparticles comprising a platelet-derived growth factor (PDGF) receptor tyrosine kinase inhibitor, especially a PDGF receptor tyrosine kinase inhibitor having a water-solubility at 20° C. between about 2.5 g/100 ml and 250 g/100 ml, more specifically nanoparticles comprising an N-phenyl-2-pyrimidine-amine derivative of formula I,

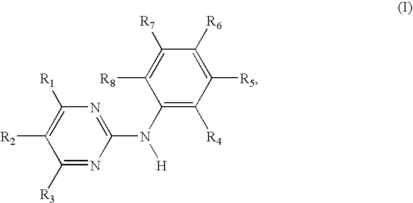

in which the symbols and substituents have the meanings as given herein above, in free form or in pharmaceutically acceptable salt form; to the intracellular delivery of PDGF receptor tyrosine kinase inhibitors such as Imatinib with bio-absorbable polymeric nanoparticles; the use of such nanoparticles in the manufacture of a pharmaceutical composition for the treatment of vascular smooth muscle cells growth diseases; to a method of treatment of warm-blooded animals suffering from vascular smooth muscle cells growth diseases; to a process to prepare such nanoparticles; to pharmaceutical compositions comprising such nanoparticles; and to drug delivery systems incorporating such nanoparticles for the prevention and treatment of vascular smooth muscle cells growth diseases.

The present invention relates to nanoparticles comprising a platelet-derived growth factor (PDGF) receptor tyrosine kinase inhibitor, especially nanoparticles comprising a N-phenyl-2-pyrimidine-amine derivative of formula I, in which the symbols and substituents have the meanings as given hereinafter, in free form or in pharmaceutically acceptable salt form; to the intracellular delivery of PDGF receptor tyrosine kinase inhibitors such as Imatinib with bio-absorbable polymeric nanoparticles; the use of such nanoparticles in the manufacture of a pharmaceutical composition for the treatment of vascular smooth muscle cells growth diseases; to a method of treatment of warm-blooded animals, including humans, suffering from vascular smooth muscle cells growth diseases; to a process to prepare such nanoparticles; to pharmaceutical compositions comprising such nanoparticles; and to drug delivery systems incorporating such nanoparticles for the prevention and treatment of vascular smooth muscle cells growth diseases.

PDGF expressed by vascular smooth muscle cells (SMCs) and monocytes, plays a central role in the pathogenesis of restenosis and atherosclerotic vascular diseases in experimental animals (Myllarniemi M, et al, Cardiovasc Drugs Ther. 1999; 13:159-68.). Atherosclerotic lesions which limit or obstruct coronary or periphery blood flow are the major cause of ischemic disease related morbidity and mortality including coronary heart disease and stroke. A number of organic compounds is known to inhibit the tyrosine kinase activity of the PDGF receptor. In particular, the mesylate salt of one of the N-phenyl-2-pyrimidine-amine derivative of formula I (see below), Imatinib mesylate (Gleevec™), is known for its capability to inhibit such PDGF receptor tyrosine kinase activity. In view of this inhibitory effect, Imatinib mesylate is currently under evaluation in clinical trials for malignant gliomas (Radford, I. R., Curr. Opin. Investig. Drugs, 3: 492-499, 2002). However, no beneficial effects of systemic administration of Imatinib against restenosis was observed in clinical studies reported by D. Zohlnhofer, et al. in J Am Coll Cardiol. 2005; 46: 1999-2003.

It was now surprisingly found that intracellular delivery of PDGF receptor tyrosine kinase inhibitors by nanoparticle technology represent an advantageous therapeutic strategy for vascular smooth muscle cells growth diseases such as restenosis, atherosclerotic vascular disease and primary pulmonary hypertension.

Hence, the present invention pertains to nanoparticles comprising a PDGF receptor tyrosine kinase inhibitor, especially nanoparticles comprising a N-phenyl-2-pyrimidine-amine derivative of formula I, in which the symbols and substituents have the meanings as given hereinafter, in free form or in pharmaceutically acceptable salt form (hereinafter referred to as NANOPARTICLES OF THE INVENTION).

In a preferred embodiment, the present invention relates to nanoparticles comprising a N-phenyl-2-pyrimidine-amine derivative of formula I,

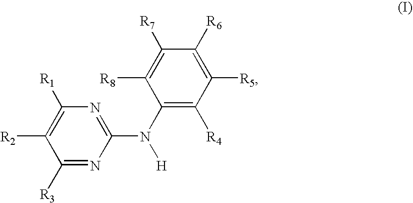

wherein
R₁ is 4-pyrazinyl; 1-methyl-1H-pyrrolyl; amino- or amino-lower alkyl-substituted phenyl, wherein the amino group in each case is free, alkylated or acylated; 1H-indolyl or 1H-imidazolyl bonded at a five-membered ring carbon atom; or unsubstituted or lower alkyl-substituted pyridyl bonded at a ring carbon atom and unsubstituted or substituted at the nitrogen atom by oxygen;
R₂ and R₃ are each independently of the other hydrogen or lower alkyl;
one or two of the radicals R₄, R₅, R₆, R₇ and R₈ are each nitro, fluoro-substituted lower alkoxy or a radical of formula II

—N(R₉)—C(═X)—(Y)ₙ—R₁₀ (II),

wherein
R₉ is hydrogen or lower alkyl,
X is oxo, thio, imino, N-lower alkyl-imino, hydroximino or O-lower alkyl-hydroximino,
Y is oxygen or the group NH,
n is 0 or 1 and
R₁₀ is an aliphatic radical having at least 5 carbon atoms, or an aromatic, aromatic—aliphatic, cycloaliphatic, cycloaliphatic-aliphatic, heterocyclic or heterocyclic-aliphatic radical,
and the remaining radicals R₄, R₅, R₆, R₇ and R₈ are each independently of the others hydrogen, lower alkyl that is unsubstituted or substituted by free or alkylated amino, piperazinyl, piperidinyl, pyrrolidinyl or by morpholinyl, or lower alkanoyl, trifluoromethyl, free, etherified or esterifed hydroxy, free, alkylated or acylated amino or free or esterified carboxy,
   or of a salt of such a compound having at least one salt-forming group.
   1-Methyl-1H-pyrrolyl is preferably 1-methyl-1H-pyrrol-2-yl or 1-methyl-1H-pyrrol-3-yl.

Amino- or amino-lower alkyl-substituted phenyl R₁ wherein the amino group in each case is free, alkylated or acylated is phenyl substituted in any desired position (ortho, meta or para) wherein an alkylated amino group is preferably mono- or di-lower alkylamino, for example dimethylamino, and the lower alkyl moiety of amino-lower alkyl is preferably linear C₁-C₃alkyl, such as especially methyl or ethyl.

1H-indolyl bonded at a carbon atom of the five-membered ring is 1H-indol-2-yl or 1H-indol-3-yl.

Unsubstituted or lower alkyl-substituted pyridyl bonded at a ring carbon atom is lower alkyl-substituted or preferably unsubstituted 2-, 4- or preferably 3-pyridyl, for example 3-pyridyl, 2-methyl-3-pyridyl or 4-methyl-3-pyridyl. Pyridyl substituted at the nitrogen atom by oxygen is a radical derived from pyridine N-oxide, i.e. N-oxido-pyridyl.

Fluoro-substituted lower alkoxy is lower alkoxy carrying at least one, but preferably several, fluoro substituents, especially trifluoromethoxy or 1,1,2,2-tetrafluoro-ethoxy.

When X is oxo, thio, imino, N-lower alkyl-imino, hydroximino or O-lower alkyl-hydroxyimino, the group C═X is, in the above order, a radical C═O, C═S, C═N—H, C═N-lower alkyl, C═N—OH or C═N—O-lower alkyl, respectively. X is preferably oxo.

n is preferably 0, i.e. the group Y is not present.

Y, if present, is preferably the group NH.

The term “lower” within the scope of this text denotes radicals having up to and including 7, preferably up to and including 4 carbon atoms.

Lower alkyl R₁, R₂, R₃ and R₉ is preferably methyl or ethyl.

An aliphatic radical R₁₀ having at least 5 carbon atoms preferably has not more than 22 carbon atoms, generally not more than 10 carbon atoms, and is such a substituted or preferably unsubstituted aliphatic hydrocarbon radical, that is to say such a substituted or preferably unsubstituted alkynyl, alkenyl or preferably alkyl radical, such as C₅-C₇alkyl, for example n-pentyl. An aromatic radical R₁₀ has up to 20 carbon atoms and is unsubstituted or substituted, for example in each case unsubstituted or substituted naphthyl, such as especially 2-naphthyl, or preferably phenyl, the substituents preferably being selected from cyano, unsubstituted or hydroxy-, amino- or 4-methyl-piperazinyl-substituted lower alkyl, such as especially methyl, trifluoromethyl, free, etherified or esterified hydroxy, free, alkylated or acylated amino and free or esterified carboxy. In an aromatic-aliphatic radical R₁₀ the aromatic moiety is as defined above and the aliphatic moiety is preferably lower alkyl, such as especially C₁-C₂alkyl, which is substituted or preferably unsubstituted, for example benzyl. A cycloaliphatic radical R₁₀ has especially up to 30, more especially up to 20, and most especially up to 10 carbon atoms, is mono- or poly-cyclic and is substituted or preferably unsubstituted, for example such a cycloalkyl radical, especially such a 5- or 6-membered cycloalkyl radical, such as preferably cyclohexyl. In a cycloaliphatic-aliphatic radical R₁₀ the cycloaliphatic moiety is as defined above and the aliphatic moiety is preferably lower alkyl, such as especially C₁-C₂alkyl, which is substituted or preferably unsubstituted. A heterocyclic radical R₁₀ contains especially up to 20 carbon atoms and is preferably a saturated or unsaturated monocyclic radical having 5 or 6 ring members and 1-3 hetero atoms which are preferably selected from nitrogen, oxygen and sulfur, especially, for example, thienyl or 2-, 3- or 4-pyridyl, or a bi- or tri-cyclic radical wherein, for example, one or two benzene radicals are annellated (fused) to the mentioned monocyclic radical. In a heterocyclic-aliphatic radical R₁₀ the heterocyclic moiety is as defined above and the aliphatic moiety is preferably lower alkyl, such as especially C₁-C₂alkyl, which is substituted or preferably unsubstituted.

Etherified hydroxy is preferably lower alkoxy. Esterified hydroxy is preferably hydroxy esterified by an organic carboxylic acid, such as a lower alkanoic acid, or a mineral acid, such as a hydrohalic acid, for example lower alkanoyloxy or especially halogen, such as iodine, bromine or especially fluorine or chlorine.

Alkylated amino is, for example, lower alkylamino, such as methylamino, or di-lower alkylamino, such as dimethylamino. Acylated amino is, for example, lower alkanoylamino or benzoylamino.

Esterified carboxy is, for example, lower alkoxycarbonyl, such as methoxycarbonyl.

A substituted phenyl radical may carry up to 5 substituents, such as fluorine, but especially in the case of relatively large substituents is generally substituted by only from 1 to 3 substituents. Examples of substituted phenyl that may be given special mention are 4-chloro-phenyl, pentafluoro-phenyl, 2-carboxy-phenyl, 2-methoxy-phenyl, 4-fluoro-phenyl, 4-cyano-phenyl and 4-methyl-phenyl.

Salt-forming groups in a compound of formula I are groups or radicals having basic or acidic properties. Compounds having at least one basic group or at least one basic radical, for example a free amino group, a pyrazinyl radical or a pyridyl radical, may form acid addition salts, for example with inorganic acids, such as hydrochloric acid, sulfuric acid or a phosphoric acid, or with suitable organic carboxylic or sulfonic acids, for example aliphatic mono- or di-carboxylic acids, such as trifluoroacetic acid, acetic acid, propionic acid, glycolic acid, succinic acid, maleic acid, fumaric acid, hydroxymaleic acid, malic acid, tartaric acid, citric acid or oxalic acid, or amino acids such as arginine or lysine, aromatic carboxylic acids, such as benzoic acid, 2-phenoxy-benzoic acid, 2-acetoxy-benzoic acid, salicylic acid, 4-aminosalicylic acid, aromatic-aliphatic carboxylic acids, such as mandelic acid or cinnamic acid, heteroaromatic carboxylic acids, such as nicotinic acid or isonicotinic acid, aliphatic sulfonic acids, such as methane-, ethane- or 2-hydroxyethane-sulfonic acid, or aromatic sulfonic acids, for example benzene-, p-toluene- or naphthalene-2-sulfonic acid. When several basic groups are present mono- or poly-acid addition salts may be formed.

Compounds of formula I having acidic groups, for example a free carboxy group in the radical R₁₀, may form metal or ammonium salts, such as alkali metal or alkaline earth metal salts, for example sodium, potassium, magnesium or calcium salts, or ammonium salts with ammonia or suitable organic amines, such as tertiary monoamines, for example triethylamine or tri-(2-hydroxyethyl)-amine, or heterocyclic bases, for example N-ethyl-piperidine or N,N′-dimethyl-piperazine.

Preference is given to nanoparticles comprising a N-phenyl-2-pyrimidine-amine derivative of formula I wherein
one or two of the radicals R₄, R₅, R₆, R₇ and R₈ are each nitro or a radical of formula II
   wherein
   R₉ is hydrogen or lower alkyl,
   X is oxo, thio, imino, N-lower alkyl-imino, hydroximino or O-lower alkyl-hydroximino,
   Y is oxygen or the group NH,
   n is 0 or 1 and
   R₁₀ is an aliphatic radical having at least 5 carbon atoms or an aromatic, aromatic—aliphatic, cycloaliphatic, cycloaliphatic-aliphatic, heterocyclic or heterocyclic-aliphatic radical,
and the remaining radicals R₄, R₅, R₆, R₇ and R₈ are each independently of the others hydrogen, lower alkyl that is unsubstituted or substituted by free or alkylated amino, piperazinyl, piperidinyl, pyrrolidinyl or by morpholinyl, or lower alkanoyl, trifluoromethyl, free, etherified or esterifed hydroxy, free, alkylated or acylated amino or free or esterified carboxy,
and the remaining substituents are as defined above.

Preference is given above all to nanoparticles comprising a N-phenyl-2-pyrimidine-amine derivative of formula I wherein
R₁ is pyridyl bonded at a carbon atom,
R₂, R₃, R₅, R₆ and R₈ are each hydrogen,
R₄ is lower alkyl,
R₇ a radical of formula II wherein
   R₉ is hydrogen,
   X is oxo,
   n is 0 and
   R₁₀ is 4-methyl-piperazinyl-methyl.

Preference is given above all to nanoparticles comprising a N-phenyl-2-pyrimidine-amine derivative of formula I which is STI571 {also known as Imatinib or N-{5-[4-(4-methylpiperazino-methyl)-benzoylamido]-2-methylphenyl}-4-(3-pyridyl)-2-pydmidine-amine}.

Very preferably, Imatinib is used in the form of its monomesylate salt. Imatinib monomesylate is very soluble in water (about 100 to 150 g/100 ml at 20° C.). Therefore, the present invention further provides NANOPARTICLES OF THE INVENTION comprising a PDGF receptor tyrosine kinase inhibitor being very soluble in water, especially having a water-solubility at 20° C. between about 2.5 g/100 ml and about 250 g/100 ml, more preferably between about 5 g/100 ml and about 175 g/100 ml, most preferably between about 75 g/100 ml and about 150 g/100 ml.

The N-phenyl-2-pyrimidine-amine derivative of formula I are generically and specifically disclosed in the U.S. Pat. No. 5,521,184 and the patent application WO 99/03854, in particular in the compound claims and the final products of the working examples. The subject-matter of the final products of the Examples and the pharmaceutical preparations are hereby incorporated into the present application by reference to these publications. Comprised are likewise the corresponding stereoisomers as well as the corresponding polymorphs, e.g. crystal modifications, which are disclosed therein. A convenient process for the manufacture of N-phenyl-2-pyrimidine-amine derivatives of formula I is disclosed in WO03/066613.

Further suitable PDGF receptor tyrosine kinase inhibitors are disclosed, for instance, in WO 98/35958, especially the compound of Example 62, and U.S. Pat. No. 5,093,330 in each case in particular in the compound claims and the final products of the working examples, the subject-matter of which are hereby incorporated into the present application by reference to these publications.

The expression “vascular smooth muscle cells growth diseases” especially relates to restenosis, atherosclerotic vascular disease and primary pulmonary hypertension.

As used herein, the term “nanoparticles” refers to particles of a mean diameter of about 2.5 nm to about 1000 nm, preferably 5 nm to about 500 nm, more preferably 25 nm to about 75 nm, and most advantageously, of between about 40 and about 50 nm. The present invention relates in particular to bio-absorbable polymeric nanoparticles comprising biodegradable polyesters.

“Biodegradable polyesters” refers to any biodegradable polyester, which is preferably synthesized from monomers selected from the group consisting of D,L-lactide, D-lactide, Llactide, D,L-lactic acid, D-lactic acid, L-lactic acid, glycolide, glycolic acid, ε-caprolactone, Ehydroxy hexanoic acid, γ-butyrolactone, y-hydroxy butyric acid, 8-valerolactone, 8-hydroxy valeric acid, hydrooxybutyric acids, malic acid and copolymers thereof.

As used herein, the term “PLGA” refers to a copolymer consisting of various ratios of lactic acid or lactide (LA) and glycolic acid or glycolide (GA). The copolymer can have different average chain lengths, resulting in different internal viscosities and differences in polymer properties.

Preferred bio-absorbable polymeric nanoparticles are poly-ethylene-glycol (PEG)modified poly-lactide-glycolide copolymer (PLGA) nanoparticles. Such nanoparticles nanoparticles with a mean diameter of 50 nm can be obtained, for instance, by applying spherical crystallization technique, e.g. as disclosed in the Examples.

As shown in the Examples below, intracellular delivery of Imatinib with bio-absorbable polymeric nanoparticle technology effectively suppresses vascular smooth muscle proliferation and migration of vascular smooth muscle cells.

In a further aspect, the present invention relates to drug delivery systems incorporating NANOPARTICLES OF THE INVENTION for the prevention and treatment of vascular smooth muscle cells growth diseases.

Many humans suffer from circulatory diseases caused by a progressive blockage of the blood vessels that perfuse the heart and other major organs. Severe blockage of blood vessels in such humans often leads to ischemic injury, hypertension, stroke or myocardial infarction. Atherosclerotic lesions which limit or obstruct coronary or periphery blood flow are the major cause of ischemic disease related morbidity and mortality including coronary heart disease and stroke. To stop the disease process and prevent the more advanced disease states in which the cardiac muscle or other organs are compromised, medical revascularization procedures such as percutaneous transluminal coronary angioplasty (PCTA), percutaneous transluminal angioplasty (PTA), atherectomy, bypass grafting or other types of vascular grafting procedures are used.

Re-narrowing (e.g. restenosis) of an artherosclerotic coronary artery after various revascularization procedures occurs in 10-80% of patients undergoing this treatment, depending on the procedure used and the aterial site. Besides opening an artery obstructed by atherosclerosis, revascularization also injures endothelial cells and smooth muscle cells within the vessel wall, thus initiating a thrombotic and inflammatory response. Cell derived growth factors such as PDGF, infiltrating macrophages, leukocytes or the smooth muscle cells themselves provoke proliferative and migratory responses in the smooth muscle cells. Simultaneous with local proliferation and migration, inflammatory cells also invade the site of vascular injury and may migrate to the deeper layers of the vessel wall.

Both cells within the atherosclerotic lesion and those within the media migrate, proliferate and/or secrete significant amounts of extracellular matrix proteins. Proliferation, migration and extracellular matrix synthesis continue until the damaged endothelial layer is repaired at which time proliferation slows within the intima. The newly formed tissue is called neointima, intimal thickening or restenotic lesion and usually results in narrowing of the vessel lumen. Further lumen narrowing may take place due to constructive remodeling, e.g. vascular remodeling, leading to further intimal thickening or hyperplasia.

Furthermore, there are also atherosclerotic lesions which do not limit or obstruct vessel blood flow but which form the so-called “vulnerable plaques”. Such atherosclerotic lesions or vulnerable plaques are prone to rupture or ulcerate, which results in thrombosis and thus produces unstable angina pectoris, myocardial infarction or sudden death. Inflamed atherosclerotic plaques can be detected by thermography.

Complications associated with vascular access devices is a major cause of morbidity in many disease states. For example, vascular access dysfunction in hemodialysis patients is generally caused by outflow stenoses in the venous circulation (Schwam S. J., et al., Kidney Int. 36: 707-711, 1989). Vascular access related morbidity accounts for about 23 percent of all hospital stays for advanced renal disease patients and contributes to as much as half of all hospitalization costs for such patients (Feldman H. I., J. Am. Soc. Nephrol. 7: 523-535, 1996). Additionally, vascular access dysfunction in chemotherapy patients is generally caused by outflow stenoses in the venous circulation and results in a decreased ability to administer medications to cancer patients. Often the outflow stenoses is so severe as to require intervention. Additionally, vascular access dysfunction in total parenteral nutrition (TPN) patients is generally caused by outflow stenoses in the venous circulation and results in reduced ability to care for these patients. Up to the present time, there has not been any effective drug for the prevention or reduction of vascular access dysfunction that accompany the insertion or repair of an indwelling shunt, fistula or catheter, such as a large bore catheter, into a vein in a mammal, particularly a human patient. Survival of patients with chronic renal failure depends on optimal regular performance of dialysis. If this is not possible (for example as a result of vascular access dysfunction or failure), it leads to rapid clinical deterioration and unless the situation is remedied, these patients will die. Hemodialysis requires access to the circulation. The ideal form of hemodialysis vascular access should allow repeated access to the circulation, provide high blood flow rates, and be associated with minimal complications. At present, the three forms of vascular access are native arteriovenous fistulas (AVF), synthetic grafts, and central venous catheters. Grafts are most commonly composed of polytetrafluoroethylene (PTFE, or Gore-Tex). Each type of access has its own advantages and disadvantages.

Vascular access dysfunction is the most important cause of morbidity and hospitalization in the hemodialysis population. Venous neointimal hyperplasia characterized by stenosis and subsequent thrombosis accounts for the overwhelming majority of pathology resulting in dialysis graft failure.

Accordingly, there is a need for effective treatment and drug delivery systems for revascularization procedure, e.g. preventing and treating intimal thickening or restenosis that occurs after injury, e.g. vascular injury, including e.g. surgical injury, e.g. revascularization-induced injury, e.g. also in heart or other grafts, for a stabilization procedure of vulnerable plaques, or for the prevention or treatment of vascular access dysfunctions.

Hence, it is also an object of this invention to provide a medical device containing NANOPARTICLES OF THE INVENTION which allows sustained delivery of the PDGF receptor tyrosine kinase inhibitor at or near the coated surfaces of the devices. In accordance with the particular findings of the present invention, there is provided:

(1) A method for preventing or treating smooth muscle cell proliferation and migration in hollow tubes (e.g. catheter-based device), or increased cell proliferation or decreased apoptosis or increased matrix deposition in a mammal in need thereof, comprising local administration of a therapeutically effective amount of PDGF receptor tyrosine kinase inhibitor employing NANOPARTICLES OF THE INVENTION.
(2) A method for the treatment of intimal thickening in vessel walls comprising the controlled delivery from any catheter-based device (e.g. indwelling shunt, fistula or catheter) or intraluminal medical device comprising NANOPARTICLES OF THE INVENTION of a therapeutically effective amount of a PDGF receptor tyrosine kinase inhibitor.
(3) A method for stabilizing vulnerable plaques in blood vessels of a subject in need of such a stabilization comprising the controlled delivery from any catheter-based device, intraluminal medical device or adventitial medical device comprising NANOPARTICLES OF THE INVENTION of a therapeutically effective amount of a PDGF receptor tyrosine kinase inhibitor.
(4) A method for preventing or treating restenosis (e.g. restenosis in diabetic patients or hypertensive patients) comprising the controlled delivery from any catheter-based device, intraluminal medical device or adventitial medical device comprising NANOPARTICLES OF THE INVENTION of a therapeutically effective amount of a PDGF receptor tyrosine kinase inhibitor.
(6) A method for the stabilization or repair of arterial or venous aneurisms in a subject comprising the controlled delivery from any catheter-based device, intraluminal medical device or adventitial medical device comprising NANOPARTICLES OF THE INVENTION of a therapeutically effective amount of a PDGF receptor tyrosine kinase inhibitor.
(7) A method for the prevention or treatment of anastomic hyperplasia in a subject comprising the controlled delivery from any catheter-based device, intraluminal medical device or adventitial medical device comprising NANOPARTICLES OF THE INVENTION of a therapeutically effective amount of a PDGF receptor tyrosine kinase inhibitor.
(8) A method for the prevention or treatment of arterial, e.g. aortic, by-pass anastomosis in a subject comprising the controlled delivery from any catheter-based device, intraluminal medical device or adventitial medical device comprising NANOPARTICLES OF THE INVENTION of a therapeutically effective amount of a PDGF receptor tyrosine kinase inhibitor.
(9) A drug delivery device or system comprising a) a medical device adapted for local application or administration in hollow tubes, e.g. a catheter-based delivery device (e.g. indwelling shunt, fistula or catheter) or a medical device intraluminal or outside of hollow tubes such as an implant or a sheath placed within the adventitia, and b) NANOPARTICLES OF THE INVENTION being releasably affixed to the catheter-based delivery device or medical device.

Such a local delivery device or system can be used to reduce the herein mentioned vascular injuries e.g. stenosis, restenosis, or in-stent restenosis, as an adjunct to revascularization, bypass or grafting procedures performed in any vascular location including coronary arteries, carotid arteries, renal arteries, peripheral arteries, cerebral arteries or any other arterial or venous location, to reduce anastomic stenosis or hyperplasia, including in the case of arterial-venous dialysis access with or without polytetrafluoroethylene or e.g. Gore-Tex grafting and with or without stenting, or in conjunction with any other heart or transplantation procedures, or congenital vascular interventions.

The local administration preferably takes place. at or near the vascular lesions sites.

The administration may be by one or more of the following routes: via catheter or other intravascular delivery system, intranasally, intrabronchially, interperitoneally or eosophagal. Hollow tubes include circulatory system vessels such as blood vessels (arteries or veins), tissue lumen, lymphatic pathways, digestive tract including alimentary canal, respiratory tract, excretory system tubes, reproductive system tubes and ducts, body cavity tubes, etc. Local administration or application of the PDGF receptor tyrosine kinase inhibitor(s) affords concentrated delivery of said PDGF receptor tyrosine kinase inhibitor(s), achieving tissue levels in target tissues not otherwise obtainable through other administration route. Additionally local administration or application may reduce the risk of remote or systemic toxicity. Preferably the smooth muscle cell proliferation or migration is inhibited or reduced according to the invention immediately proximal or distal to the locally treated or stented area.

Means for local delivery of the PDGF receptor tyrosine kinase inhibitor(s) to hollow tubes can be by physical delivery of the NANOPARTICLES OF THE INVENTION either internally or externally to the hollow tube. Local delivery includes catheter delivery systems, local injection devices or systems or indwelling devices. Such devices or systems would include, but not be limited to, indwelling shunt, fistula, catheter, stents, endolumenal sleeves, stent-grafts, controlled release matrices, polymeric endoluminal paving, or other endovascular devices, embolic delivery particles, cell targeting such as affinity based delivery, internal patches around the hollow tube, external patches around the hollow tube, hollow tube cuff, external paving, external stent sleeves, and the like. See, Eccleston et al. (1995) Interventional Cardiology Monitor 1:33-40-41 and Slepian, N.J. (1996) Intervente. Cardiol. 1:103-116, or Regar E, Sianos G, Serruys P W. Stent development and local drug delivery. Br Med Bull 2001, 59:227-48 which disclosures are herein incorporated by reference. Preferably the delivery device or system fulfils pharmacological, pharmacokinetic and mechanical requirements. Preferably it also is suitable for sterilization.

The stent according to the invention can be any stent, including self-expanding stent, or a stent that is radially expandable by inflating a balloon or expanded by an expansion member, or a stent that is expanded by the use of radio frequency which provides heat to cause the stent to change its size.

Delivery or application of the PDGF receptor tyrosine kinase inhibitor(s) can occur using indwelling shunt, fistula, stents or sleeves or sheathes. A stent composed of or coated with a polymer or other biocompatible materials, e.g. porous ceramic, e.g. nanoporous ceramic, into which the NANOPARTICLES OF THE INVENTION have been impregnated or incorporated can be used. Such stents can be biodegradable or can be made of metal or alloy, e.g. Ni and Ti, or another stable substance when intented for permanent use. The NANOPARTICLES OF THE INVENTION may also be entrapped into the metal of the stent or graft body which has been modified to contain micropores or channels. Also lumenal and/or ablumenal coating or external sleeve made of polymer or other biocompatible materials, e.g. as disclosed above, that contain the NANOPARTICLES OF THE INVENTION can also be used for local delivery of PDGF receptor tyrosine kinase inhibitor(s).

By “biocompatible” is meant a material which elicits no or minimal negative tissue reaction including e.g. thrombus formation and/or inflammation.

For example, the NANOPARTICLES OF THE INVENTION may be incorporated into or affixed to the stent (or to indwelling shunt, fistula or catheter) in a number of ways and utilizing any biocompatible materials; it may be incorporated into e.g. a polymer or a polymeric matrix and sprayed onto the outer surface of the stent. A mixture of the NANOPARTICLES OF THE INVENTION and the polymeric material may be prepared in a solvent or a mixture of solvents and applied to the surfaces of the stents also by dipcoating, brush coating and/or dip/spin coating, the solvent (s) being allowed to evaporate to leave a film with entrapped drug(s). In the case of stents where the PDGF receptor tyrosine kinase inhibitor(s) is delivered from micropores, struts or channels, a solution of a polymer may additionally be applied as an outlayer to control the release of the PDGF receptor tyrosine kinase inhibitor(s); alternatively, the NANOPARTICLES OF THE INVENTION may be comprised in the micropores, struts or channels and the adjunct may be incorporated in the outlayer, or vice versa. The NANOPARTICLES OF THE INVENTION may also be affixed in an inner layer of the stent (or of the indwelling shunt, fistula or catheter) and the adjunct in an outer layer, or vice versa. The NANOPARTICLES OF THE INVENTION may also be attached by a covalent bond, e.g. esters, amides or anhydrides, to the stent (or of the indwelling shunt, fistula or catheter) surface, involving chemical derivatization. The NANOPARTICLES OF THE INVENTION may also be incorporated into a biocompatible porous ceramic coating, e.g. a nanoporous ceramic coating.

Examples of polymeric materials include hydrophilic, hydrophobic or biocompatible biodegradable materials, e.g. polycarboxylic acids; cellulosic polymers; starch; collagen; hyaluronic acid; gelatin; lactone-based polyesters or copolyesters, e.g. polylactide; polyglycolide; polylactide-glycolide; polycaprolactone; polycaprolactone-glycolide; poly(hydroxybutyrate); poly(hydroxyvalerate); polyhydroxy(butyrate-co-valerate); polyglycolide-co-trimethylene carbonate; poly(diaxanone); polyorthoesters; polyanhydrides; polyaminoacids; polysaccharides; polyphosphoeters; polyphosphoester-urethane; polycyanoacrylates; polyphosphazenes; poly(ether-ester) copolymers, e.g. PEO-PLLA, fibrin; fibrinogen; or mixtures thereof; and biocompatible non-degrading materials, e.g. polyurethane; polyolefins; polyesters; polyamides; polycaprolactame; polyimide; polyvinyl chloride; polyvinyl methyl ether; polyvinyl alcohol or vinyl alcohol/olefin copolymers, e.g. vinyl alcohol/ethylene copolymers; polyacrylonitrile; polystyrene copolymers of vinyl monomers with olefins, e.g. styrene acrylonitrile copolymers, ethylene methyl methacrylate copolymers; polydimethylsiloxane; poly(ethylene-vinylacetate); acrylate based polymers or copolymers, e.g. polybutylmethacrylate, poly(hydroxyethyl methylmethacrylate); polyvinyl pyrrolidinone; fluorinated polymers such as polytetrafluoethylene; cellulose esters e.g. cellulose acetate, cellulose nitrate or cellulose propionate; or mixtures thereof.

According to the method of the invention or in the device or system of the invention, the PDGF receptor tyrosine kinase inhibitor(s) may elute passively, actively or under activation, e.g. light-activation.

It can be shown by established test models and especially those test models described herein that the NANOPARTICLES OF THE INVENTION, are suitable to be used in an effective prevention or treatment of vascular smooth muscle cells (SMCs) growth diseases.

As shown in the Examples, when incubated with rat aortic and human coronary artery arterial vascular SMCs, nanoparticles loaded with a fluorescence marker instead of a PGDF receptor tyrosine kinase inhibitor enter rapidly into almost all SMCs and reach the peri-nuclear region within 1 hour. In addition, such nanoparticles incorporated into the cells show prolonged retention in the cytoplasm at least for 14 days. As further shown in the Examples, non-encapsulated Imatinib at 0.1, 1.0, and 10 μM inhibit PDGF-induced proliferation/migration of SMCs in a dose-dependent manner: Imatinib at 0.1 μM shows no effect, but Imatinib at 10 μM normalizes the PDGF-induced response. Co- or pre-treatment with nanoparticles containing Imatinib at 0.1 μM completely normalizes PDGF-induced proliferation/migration of SMCs. This demonstrates that the inhibitory potency of nanoparticulated Imatinib is at least 100-times stronger, compared with that of non-encapsulated free Imatinib.

### SHORT DESCRIPTION OF THE FIGURES

FIG. 1A:

When incubated for 30 minutes with rat aortic and human coronary arterial SMCs, the coumarin-6 loaded PEG-PLGA nanoparticles show excellent capability of passing through cellular membrane and reaching to peri-nuclear region. Nuclear is counterstained with propidium iodide (PI). Scale=50 g/m. A large fraction of the nanoparticles rapidly enters into the cells: the delivery rate is about 60% at 15 min of passing through the cellular membrane and reaching the peri-nuclear region within 1 hour.

FIG. 1B: Efficiency of Cellular Uptake of PEG-PLGA Nanoparticles

Cellular uptake is observed independently of concentrations of PEG-PLGA nanoparticles suspension. Cellular uptake percentage was quantified by measuring fluorescence positive areas/cellular surface areas×100 with a computer-assisted microscope. Data are mean±SEM (n=4).

PDGF-BB induced SMCs proliferation and migration is inhibited with Imatinib and Imatinib loaded PEG-PLGA nanoparticles

FIG. 2A:

Stimulation of human coronary arterial vascular SMCs with 10 ng/ml PDGF-BB causes a significant increase in cell number. Imatinib dose-dependently reduces the SMCs proliferation induced by PDGF-BB. A concentration of 10 μM Imatinib completely abolishes the stimulatory effect of PDGF-BB on cell proliferation. In contrast, simultaneously or pretreated treated cells with 0.5 mg/ml Imatinib loaded PEG-PLGA nanoparticles (containing 0.1 μM Imatinib) attenuate PDGF-BB induced proliferation. Data are mean±SEM (n=6). *P<0.01 vs control, P<0.01 vs PDGF.

FIG. 2B

Migration of rat aortic SMCs induced by PDGF-BB is measured in the Transwell migration chamber. Imatinib exhibit a dose-dependent inhibitory effect on PDGF-BB dependent migration. Similar to proliferation assay results, cells simultaneously treated or pretreated with 0.5 mg/ml Imatinib loaded PEG-PLGA nanoparticles (containing 0.1 μM Imatinib) attenuate PDGF-BB induced proliferation.

FIG. 3

MTS assay for PEG-PLGA nanoparticles cytotoxicity. Bargraph shows the viability of human coronary arterial vascular SMCs incubated with indicated concentration of FITC loaded PEG-PLGA nanoparticles for 48 hours. Data are mean±SEM (n=5).

PDGF-induced proliferation and migration of SMCs are completely normalized by pretreatment with nanoparticles containing low concentrations (0.1 μM) of Imatinib. In contrast, similar dose range of free Imatinib shows no effects. The inhibitory potency of nanoparticulated Imatinib is 100-times stronger compared with that of free Imatinib.

FIG. 4A

Intra-stent stenosis (neointimal thickening) inhibiting effect by Imatinib loaded PEG-PLGA nanoparticles. Bargraph shows the stent to artery ratio. BM means bare metal, and NP means nanoparticle. Data are mean±SEM.

FIG. 4B

Lumen stenosis inhibiting effect by Imatinib loaded PEG-PLGA nanoparticles. Bargraph shows the angiographical stenosis (%). BM means bare metal, and NP means nanoparticle. The drug is Imatinib. Data are mean±SEM.

FIG. 5A Suppression of neointimal formation following vascular injury by Imatinib nanoparticle. Bargraph shows the neointimal area of graft vessel treated with indicated reagent for 30 minutes. Data are mean±SEM. * represents p<0.05 vs no treatment.

FIG. 5B

Suppression of lumen stenosis following vascular injury by Imatinib nanoparticle. Bargraph shows the lumen stenosis rate of graft vessel treated with indicated reagent for 30 minutes. Data are mean±SEM. * represents that p<0.05 vs no treatment.

### DETAILED DISCUSSION OF THE EXAMPLES

### Cell Uptake and Intracellular Distribution of Nanoparticles

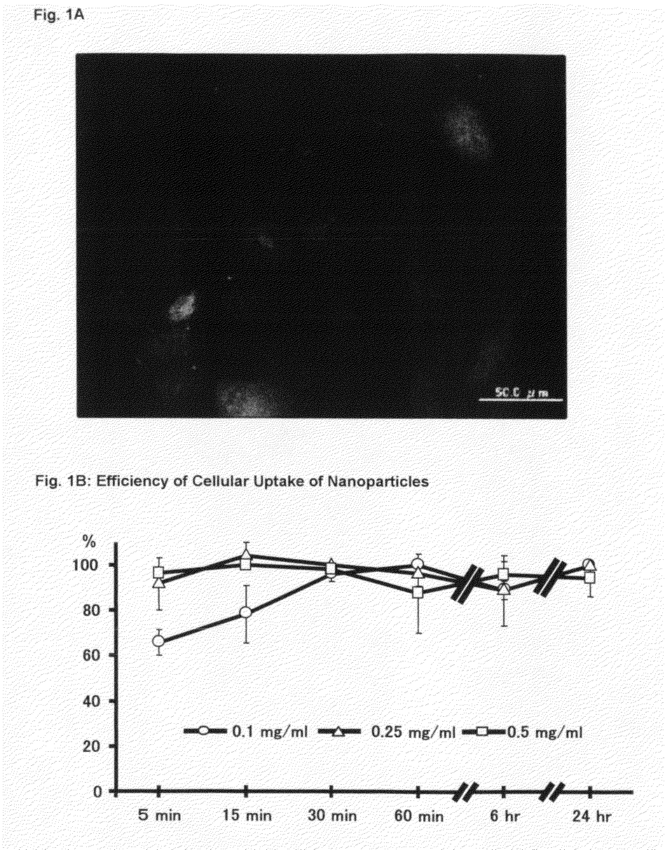

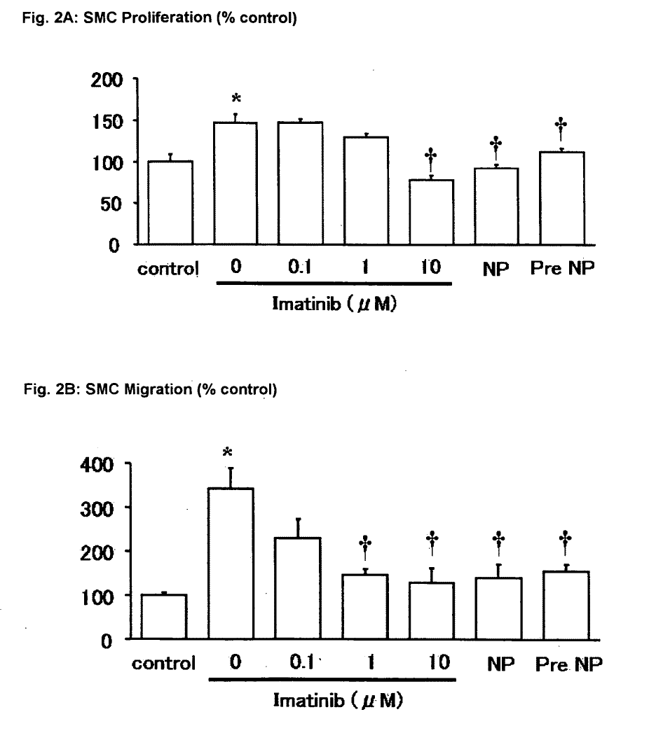

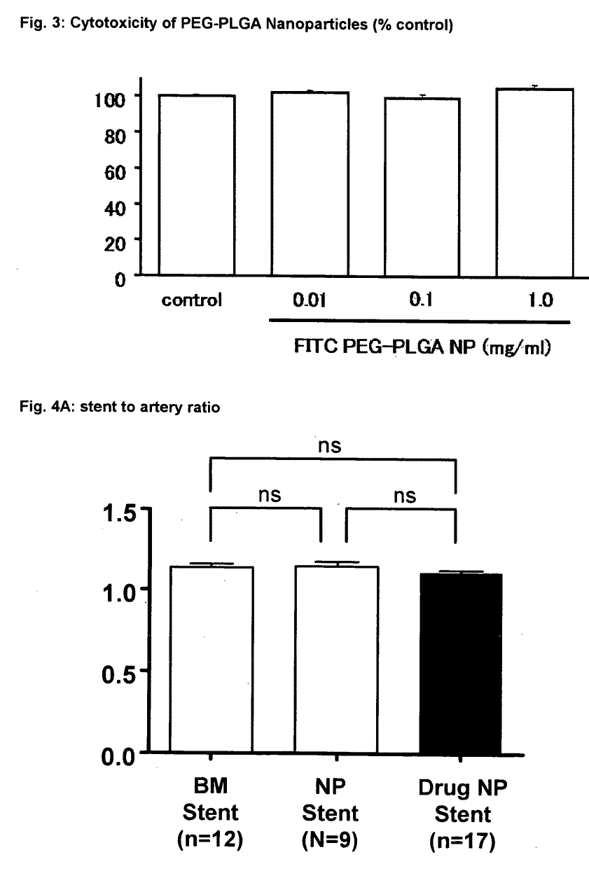

Fluorescent labeling makes cellular uptake of nanoparticles readily detectable by fluorescence microscopy. It was found that when incubated with rat aortic and human coronary artery arterial SMCs, the fluorescence encapsulated nanoparticles show excellent capacity of intracellular delivery (FIG. 1). In contrast, no fluorescence was detected when the SMCs are incubated with blank nanoparticles or fluorescence only. A large fraction (>90%) of the nanoparticles rapidly enter into the cells, and incorporation rate sustain to be stable until 24 hours (FIG. 2); delivery rates are about 100% at 15 min, 98**% at 30 min, 88**% at 60 min, 96% at 6 hours, and 94% at 24 hours when cells are incubated with PEG-PLGA nanoparticle at 0.5 mg/mL. The cells are viable during the course of this study. Concerning the time course of incorporation of the nanoparticles by SMCs it was found that the nanoparticles are uptaken through endocytosis pathway and remain stable in the cytoplasm especially in the perinuclear regions. Long-term trace study show that the discrete pattern of fluorescence remains intact around the nucleus until 14 days after incubation of the nanoparticles for 30 minutes and wash.

PDGF-BB Induced SMCs Proliferation and Migration is Inhibited with Imatinib and Imatinib Loaded PEG-PLGA Nanoparticles

It was further found that stimulation of human coronary artery arterial vascular SMCs with 10 ng/ml PDGF-BB at 10 ng/ml causes a significant increase in cell number. Free Imatinib reduces the SMCs proliferation induced by PDGF-BB in a dose-dependent manner. A concentration of 10 μM Imatinib completely abolishes the stimulatory effect of PDGF-BB-induced on cell proliferation. In contrast, both co-treatment and pre-treatment with the 0.5 mg/ml Imatinib loaded PEG-PLGA nanoparticles (containing 0.1 μM Imatinib) attenuate PDGF-BB induced proliferation to the similar extent as does free Imatinib at 10 μM. With other words, the magnitudes of the inhibition are comparable between free Imatinib at 10 μM and nanoparticulated Imatinib at 0.1 μM.2A).

Finally, it was found that PDGF-BB-induced migration is also inhibited by free Imatinib in rat aortic SMCs. Imatinib exhibits a dose-dependent manner in rat SMCs. Both co-treatment and pre-treatment with the PEG-PLGA nanoparticles containing 0.1 μM Imatinib prevent PDGF-BB induced migration to the similar extent as did free Imatinib at 1 μM. That is, the magnitudes of the inhibition are comparable between free Imatinib at 1 μM and nanoparticulated Imatinib at 0.1 μM. Similar to the proliferation assay results, simultaneously or pretreated treated cells with 0.5 mg/ml Imatinib loaded PEG-PLGA nanoparticles (containing 0.1 μM Imatinib) attenuate PDGF-BB induced proliferation.

PDGF-induced proliferation and migration of SMCs are completely normalized by pretreatment with nanoparticles containing low concentrations (0.1 μM) of Imatinib. In contrast, similar dose range of free Imatinib show no effects. The inhibitory potency of nanoparticulated Imatinib is 100-times stronger compared with that of free Imatinib.

In accordance with the particular findings of the invention, the present invention also provides a method for the treatment of warm-blooded animals, including humans, in which a therapeutically effective dose of NANOPARTICLES OF THE INVENTION is administered to such a warm-blooded animal suffering from vascular smooth muscle cells growth diseases.

The present invention relates also to a pharmaceutical composition comprising NANOPARTICLES OF THE INVENTION, especially for the treatment of vascular smooth muscle cells growth diseases.

The NANOPARTICLES OF THE INVENTION are up taken similarly by other cell types such as endothelial cells, leukocytes, cardiac myocytes and fibroblasts, which allows to apply the NANOPARTICLES OF THE INVENTION to several treatment-intractable diseases. Therefore, in a broader aspect of the present invention, the NANOPARTICLES OF THE INVENTION can also be used for the treatment of atherosclerosis (myocardial infarction, brain infarction, peripheral artery disease), vein graft failure, post-transplant arteriosclerosis, organ fibrosis and arterial aneurysm.

Pharmaceutical compositions comprising NANOPARTICLES OF THE INVENTION together with pharmaceutically acceptable carriers that are suitable for topical, enteral, for example oral or rectal, or parenteral administration, and may be inorganic or organic, solid or liquid. For oral administration there are used especially tablets or gelatin capsules comprising the NANOPARTICLES OF THE INVENTION together with diluents, for example lactose, dextrose, sucrose, mannitol, sorbitol, cellulose and/or glycerol, and/or lubricants, for example silicic acid, talc, stearic acid or salts thereof, such as magnesium or calcium stearate, and/or polyethylene glycol and/or stabilizers. Tablets may also comprise binders and, if desired, disintegrators, adsorbents, dyes, flavourings and sweeteners. The NANOPARTICLES OF THE INVENTION can also be used in the form of parenterally administrable compositions or in the form of infusion solutions. Such solutions comprise excipients, for example stabilizers, preservatives, welting agents and/or emulsifiers, salts for regulating the osmotic pressure and/or buffers. The present pharmaceutical compositions are prepared in a manner known per se, and comprise approximately from 1% to 100%, especially from approximately 1% to approximately 20%, active ingredient.

The dosage range of the NANOPARTICLES OF THE INVENTION to be employed depends upon factors known to the person skilled in the art including species of the warm-blooded animal, body weight and age, the mode of administration, the particular substance to be employed and the status of the disease to be treated. Unless stated otherwise herein, NANOPARTICLES OF THE INVENTION are preferably administered from one to four times per day.

The following Examples serve to illustrate the invention without limiting the invention in its scope.

### Example 1

### Preparation of Nanoparticles

Fluorescence marker or Imatinib loaded PEG-PLGA nanoparticles are prepared by the solvent diffusion method. Hydrophobic poly (D, L-lactic-co-glycolic acid) (PLGA) with L:G molar ratio of 75:25 and MW of 20000, polyvinylalcohol (PVA) with MW of 30,000-70,000, fluorescence marker coumarin-6, are dissolved in ethyl acetate. Hydrosoluble polyethylene glycol (PEG with an average molecular weight ranging from 2,000 to 20,000 purchased from Aldrich Chemical Co) is first dissolved in water and then emulsified in the PLGA dissolving organic phase. An oil phase solution of PEG-PLGA is slowly poured into an aqueous solution containing PVA and emulsified using a microtip probe sonicator. The PEG-PLGA copolymer solution also contained 0.05% (w/v) coumarin-6 or 5% (w/v) fluoresceine isothiocyanate (FITC) as fluorescence marker or 15% (w/v) Imatinib, for the preparation of fluorescence marker or Imatinib loaded PEG-PLGA nanoparticles, respectively. The resulted oil-in-water emulsion is then stirred at room temperature. The obtained PEG-PLGA nanoparticles are collected by centrifugation and washed with Millipore water for 3 times to remove excessive emulsifier.

### Example 2

### Fluorescence Microscopy

Rat aortic SMCs (Toyobo) are cultured in DMEM (Sigma) supplemented with 10% FBS (Equitech-Bio, Inc.) except where otherwise indicated. Human coronary artery SMCs (Cambrex Bio Science Walkersville, Inc.) are cultured in SmGM-2 (Cambrex Bio Science). Each Cells are used between passages 4 to 8. Rat aortic SMCs are seeded on chambered cover glasses and incubated at 37° C./5% CO₂ environment until cells are subconfluent. On the day of experiment, the growth medium is replaced with the coumarin-6 loaded PEG-PLGA nanoparticles suspension medium (0.5 mg/ml) and then further incubated for 1 hour. At the end of experiment, the cells are washed three times with PBS to eliminate excess nanoparticles which are not incorporated into the cells. Then, the cells are fixed with 1% formaldehyde/PBS buffer and nuclear is counterstained with propidium iodide (PI). Cellular uptake of coumarin-6 loaded PEG-PLGA nanoparticles is evaluated by fluorescence microscopy.

Alternatively, rat aortic SMCs are incubated with FITC loaded PEG-PLGA nanoparticles (0.5 mg/ml) for 30 minutes. Then, the medium is discarded and washed three times with PBS and followed by incubation with fresh medium. Thereafter, the cells are observed for 14 days.

### Example 3

### Cellular Uptake and Intracellular Distribution of Nanoparticles

Rat aortic SMCs are seeded on 48-well culture plate to an initial concentration of 1×105 cells per well (n=4 per well). The coumarin-6 loaded PEG-PLGA nanoparticles suspension medium is added to the cells at final concentration ranging from 0.1 to 0.5 mg/ml. To examine the effects of incubation time on intracellular uptake, the duration is varied from 5 minutes to 24 hours. At different time points, the nanoparticle-containing medium is removed, and the cells are washed three times with PBS. The cells are fixed with 1% formaldehyde/PBS buffer. Differential interference contrast (DIC) and fluorescence images are captured with a microscope. The images are digitized and analyzed with Adobe Photoshop and Scion Image Software. The total number of fluorescence positive cells in each field and the number of total cells was counted. Cellular uptake percentage was assessed by the percentage of fluorescence positive cells per total cells in each field. Cellular uptake percentage is assessed by the following formula; fluorescence positive areas/cellular surface areas×100.

### Example 4

### SMC Proliferation Assay

Human coronary artery arterial vascular SMCs (Cambrex Bio Science Walkersville, Inc) are seeded on 48-well culture plates (FALCON 354506 BIOCOAT CELL WARE Human Fibronectin) at 5×10³ cells per well (n=6 per group) in SM-BM with 10% FBS. After 24 hours, the cells are starved for 72 hours in serum free medium to obtain quiescent non-dividing cells. After starvation, recombinant PDGF-BB (Sigma) 10 ng/m¹ is added. Also, various concentration of Imatinib (0.1, 1, 10 μM) or Imatinib loaded PEG-PLGA nanoparticles (0.5 mg/ml) are added to each well. In some experiments, Imatinib loaded PEG-PLGA nanoparticles (0.5 mg/ml) are added to the cells in the last 24 hour. These wells are washed with PBS before PDGF stimulation. Four days later, the cells are fixed with methanol and stained with Diff-Quick staining solution (Baxter). A single observer who is blinded the experimental protocol counted the number of cells/plate under a microscope for quantification of SMC proliferation. Imatinib loaded PEG-PLGA nanoparticles (0.5 mg/ml) is corresponding to 0.1 μM concentrations of free Imatinib.

### Example 5

### SMC Migration Assay

Migration of rat aortic SMCs is assessed with a Boyden chamber type cell migration assay kit housing a collagen-precoated polycarbonate membrane with 8.0-μm pores (Chemicon), as we previously described (Ono H, Ichiki T, et al. Arterioscler Thromb Vasc Biol. 2004; 24:1634-9.). SMCs are grown to semiconfluent and then made quiescent in serum free medium for 24 hours before migration. The cells (1×10⁵ cells/ml) are added to the upper chamber of the membrane (n=6 per group) and allowed to migrate through the pores. The cells are allowed 30 minutes to attach to the membrane before addition of Imatinib (0.1, 1, 10 μM) or Imatinib loaded PEG-PLGA nanoparticles (0.5 mg/ml). In some experiments, Imatinib loaded PEG-PLGA nanoparticles (0.5 mg/ml) are added to the cells in last 24 hour. These cells are washed with PBS before PDGF stimulation. SMCs are then exposed. to PDGF-BB (10 ng/ml) in the lower chamber for 4 hours, after which non-migrated cells are removed from the upper chamber using a cotton swab. The SMCs that migrate to the lower side of the filter are fixed in methanol, stained with Diff-Quick staining solution (Baxter), and counted under a microscope for quantification of SMC migration.

### Example 6

### Preparation of Cationic PLGA NP with Surface Modification with Chitosan

A lactide/glycolide copolymer (PLGA) with an average molecular weight of 20,000 and a copolymer ratio of lactide to glycolide of 75:25 (Wako, Osaka, Japan) was used as a wall material for the nanospheres. Fluorescein-isothiocyanate (FITC, Dojin Chemical, Tokyo, Japan) was used as a fluorescent marker of the nanospheres. Chitosan (Mw. 50,000; deacetylation degree 80%; Katakura chikkarin, Tokyo, Japan) was used to coat the surface of PLGA NP.

Polyvinylalcohol (PVA-403; Kuraray; Osaka, Japan) was used as a dispersing agent. Caprylate and caprate triglyceride (Triester R F-810; Nikko Chemicals, Tokyo, Japan) was used as a nontoxic oil-dispersing medium because of its good biocompatibility and low viscosity. Hexaglycerin-condensed ricinoleate (HGCR; hexaglyn PR-15; Nikko Chemicals, Tokyo, Japan) and sorbitan monooleate (SpanR 80; Kishida Chemicals, Tokyo, Japan) were employed as nontoxic emulsifiers for pulmonary administration. Imatinib (a PDGF-R tyrosine kinase inhibitor, Novartis) was purchased from pharmacy.

PLGA NP incorporated with FITC or imatinib were prepared by a previously reported emulsion solvent diffusion method in oil. PLGA (100 mg) were dissolved in a mixture of acetone (3 ml), methanol (2 ml) and Span 80 (100 mg). Then, FITC or imatinib were added into this solution. The resultant polymer-FITC or -drug solution was emulsified in an n-hexane (40 ml) Triester F-810 (60 ml) mixture containing 1.2% w/w HGCR under stirring at 400 rpm using the propeller-type agitator with three blades. After agitating the system for 3 h under reduced pressure at 35° C., the entire suspension was added to n-hexane (20 ml) and centrifuged (43,400×g for 10 min at 4° C.), and then the process was duplicated. The sediment was then incubated in 21 ml of mixed aqueous solution of 1% PVA (20 ml) and 1% chitosan (1 ml) for 5 min. After centrifugation, the unencapsulated reagent and the unbound polymer were removed by rinsing the sediment with distilled water. After repeating this process, the resultant dispersion was freeze-dried under the same conditions.

The FITC- and imatinib-incorporated PLGA nanoparticles contained 5% (w/v) FITC and 10% (w/v) imatinib, respectively. The zeta potential of the nanospheres as measured by a laser particle analyzer (LPA 3100; Otsuka Electronics, Osaka, Japan) was 21.2 mV±3.1 at pH 4.4. The average particle diameter of the nanospheres was 200 nm by Microtrack UPA150 (Nikkiso, Tokyo, Japnan).

### Example 7

### Preparation of NP-Eluting Stent by a Cationic Electrodeposit Coating Technology

A 15-mm-long stainless-steel, balloon-expandable stents (Multilink, Guidant) were ultrasonically cleaned by acetone, ethanol (70%), and Milli Q. Cationic electrodeposite coating was prepared on cathodic stents in PLGA NP solution at a concentrations of 2.5 mg/mL in Milli Q water with current maintained at 2.0 mA by a direct current power supply (DC power supply, Nippon Stabilizer Co, Tokyo, Japan) for different period under sterile conditions. The coated stents were then rinsed with Milli Q water and suction dried overnight at 1 mmHg. Some coating stents were observed with scanning electron microscopy (JXM8600, JEOL, Tokyo, Japan) pre- and post-balloon expansion.

As control, dip-coated stents with thin layers of PLGA polymer containing FITC were prepared (coating amount of PLGA and FITC was adjusted to be same as the NP eluting stent) as we previously described. Prior to experimental use, all stents were dried vacuously and sterilized using ethylene oxide gas.

### Example 8

### The Effect of the Imatinib Nanoparticle Coated Stent

Imatinib (10% w/v) loaded cationic nanoparticles and drug-free cationic nanoparticles are prepared as described in Example 6. A surface of a metal stent is coated respectively by each of these nanoparticles using an electrodeposition coating technique as described in Example 7. The Imatinib loaded nanoparticle coating stent (Drug NP stent) and drug-free nanoparticle stent (NP stent) and bare metal stent (BM sent, as control) are mounted in a balloon respectively, which are implanted into a porcine coronary artery. After 4 weeks, a coronary angiography is performed to evaluate an intra-stent stenosis (neointimal thickening). A quantitative coronary angiography method is employed to determine a degree of a lumen stenosis (angiographic stenosis %).

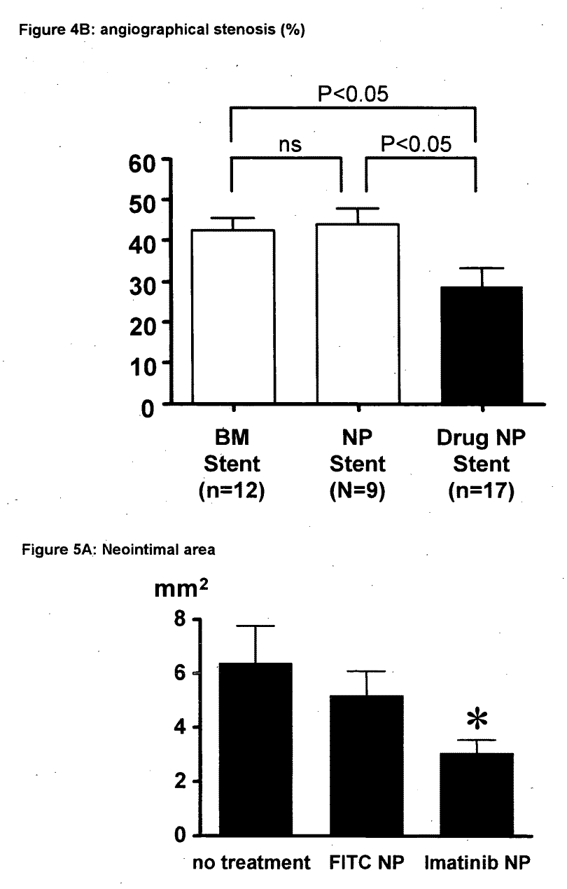

The degree of an expansion of the stent or the degree of a vascular injury (stent-to artery ratio) are comparable among those three groups with no significant differences (FIG. 4A). However, the degree of lumen stenosis is significantly decreased with the Imatinib loaded nanoparticle coating stent group. On the other hand, the suppressor effect on the neointimal formation can not find in a stent group coated by Imatinib using only polymer (FIG. 4B). Therefore, the Imatinib loaded nanoparticle coating stent is found to be effective against neointimal thickening.

### Example 9

### Suppression of Neointimal Formation Following Vascular Injury by Imatinib Nanoparticle

A rabbit vein autograft is implanted into a carotid artery to prepare a rabbit vein graft failure model. In this model, a lumen stenosis due to neointimal formation develops after 4 weeks. Four groups consisting of a non-treated control vein-graft, a vein-graft treated by a Imatinib-free (FITC) nanoparticle for 30 min., a vein-graft treated by a Imatinib loaded nanoparticle for 30 min., a vein-graft treated with Imatinib only for 30 min. (concentrations of Imatinib in Group 3 and 4 are 10% w/v) are prepared to study whether a delivery of Imatinib to the vein-graft by nanoparticle is effective or not.

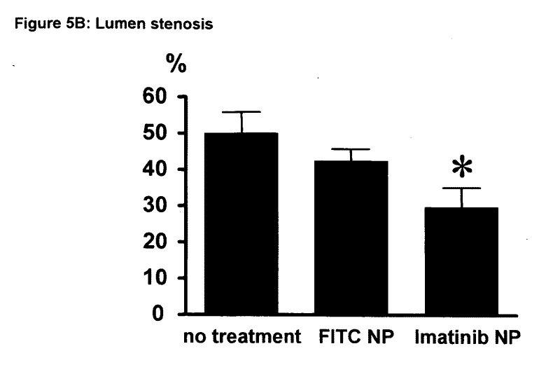

For two groups, the non-treated control vein-graft and the vein-graft treated by the Imatinib-free nanoparticle for 30 min., develop vein graft failure (neointimal formation) as previously reported. The degrees of neointimal formation are comparable between these two groups. On the other hand, in the Imatinib loaded nanoparticle group, the formation of neointimal is significantly suppressed. No suppression is observed with the group treated with Imatinib only (FIG. 5A). Therefore, it is found that the delivery of Imatinib into the vascular wall cells by the Imatinib nanoparticle are effective for treat vein graft failure, in particular Lumen stenosis (FIG. 5B).

1. Nanoparticles comprising a PDGF receptor tyrosine kinase inhibitor.

2. Nanoparticles according to claim 1 the PDGF receptor tyrosine kinase inhibitor having a water-solubility at 20° C. between about 2.5 g/100 ml and 250 g/100 ml.

3. Nanoparticles according to claim 1 wherein the PDGF receptor tyrosine kinase inhibitor is a N-phenyl-2-pyrimidine-amine derivative of formula I 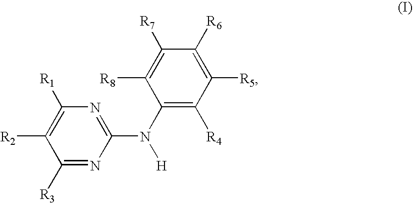
wherein
R₁ is 4-pyrazinyl; 1-methyl-1H-pyrrolyl; amino- or amino-lower alkyl-substituted phenyl, wherein the amino group in each case is free, alkylated or acylated; 1H-indolyl or 1H-imidazolyl bonded at a five-membered ring carbon atom; or unsubstituted or lower alkyl-substituted pyridyl bonded at a ring carbon atom and unsubstituted or substituted at the nitrogen atom by oxygen;
R₂ and R₃ are each independently of the other hydrogen or lower alkyl;
one or two of the radicals R₄, R₅, R₆, R₇ and R₈ are each nitro, fluoro-substituted lower alkoxy or a radical of formula II
—N(R₉)—C(═X)—(Y)ₙ—R₁₀ (II),
wherein
R₉ is hydrogen or lower alkyl,
X is oxo, thio, imino, N-lower alkyl-imino, hydroximino or O-lower alkyl-hydroximino,
Y is oxygen or the group NH,
n is 0 or 1 and
R₁₀ is an aliphatic radical having at least 5 carbon atoms, or an aromatic, aromatic—aliphatic, cycloaliphatic, cycloaliphatic-aliphatic, heterocyclic or heterocyclic-aliphatic radical,
and the remaining radicals R₄, R₅, R₆, R₇ and R₈ are each independently of the others hydrogen, lower alkyl that is unsubstituted or substituted by free or alkylated amino, piperazinyl, piperidinyl, pyrrolidinyl or by morpholinyl, or lower alkanoyl, trifluoromethyl, free, etherified or esterifed hydroxy, free, alkylated or acylated amino or free or esterified carboxy,
or a salt of such a compound having at least one salt-forming group.

4. Nanoparticles according to claim 3 wherein the N-phenyl-2-pyrimidine-amine derivative of formula I is N-{5-[4-(4-methyl-piperazino-methyl)-benzoylamido]-2-methylphenyl}-4-(3-pyridyl)-2-pyrimidine-amine} (Imatinib).

5. Nanoparticles according to claim 4, wherein Imatinib is used in the form of its monomesylate salt.

6. Nanoparticles according to claim 1, wherein the nanoparticles have a mean diameter of about 2.5 nm to about 1000 nm.

7. Nanoparticles according to claim 1, wherein the nanoparticles have a mean diameter of about 5 nm to about 500 nm.

8. Nanoparticles according to claim 1, wherein the nanoparticles comprise biodegradable polyesters.

9. Nanoparticles according to claim 1, wherein the nanoparticles comprise poly-ethylene-glycol (PEG)-modified poly-lactide-glycolide copolymer (PLGA) nanoparticles.

10. A process for the preparation of nanoparticles according to claim 1 with a mean diameter of 50 nm by applying spherical crystallization technique.

11. A method for the treatment of warm-blooded animals, including humans, in which a therapeutically effective dose of nanoparticles according to claim 1 is administered to such a warm-blooded animal suffering from vascular smooth muscle cells growth diseases.

12. The use of nanoparticles according to claim 1 for the manufacture of a pharmaceutical composition for the treatment of vascular smooth muscle cells growth diseases.

13. The method of claim 11 wherein the vascular smooth muscle cells growth diseases is selected from restenosis, atherosclerotic vascular disease and primary pulmonary hypertension.

14. A pharmaceutical composition comprising nanoparticles according to claim 1.

15. Use of nanoparticles according to claim 1 for the manufacture of a pharmaceutical product for stabilizing vulnerable plaques in blood vessels of a subject in need of such a stabilization, for preventing or treating restenosis in diabetic patients, or for the prevention or reduction of vascular access dysfunction in association with the insertion or repair of an indwelling shunt, fistula or catheter in a subject in need thereof.

16. A method for the prevention or reduction of vascular access dysfunction in association with the insertion or repair of an indwelling shunt, fistula or catheter into a vein or artery, or actual treatment, in a mammal in need thereof, which comprises administering to the subject an effective amount of nanoparticles according to claim 1.

17. Use or method according to claim 15 for use in dialysis patients.

18. A drug delivery device or system comprising i) a medical device adapted for local application or administration in hollow tubes and ii) nanoparticles according to claim 1 being releasably affixed to the drug delivery device or system.

19. A method for the treatment of intimal thickening in vessel walls comprising the controlled delivery of a therapeutically effective amount of a PDGF receptor tyrosine kinase inhibitor from any catheter-based device or intraluminal medical device comprising nanoparticles according to claim 1.

20. A method for stabilizing vulnerable plaques in blood vessels of a subject in need of such a stabilization comprising the controlled delivery of a therapeutically effective amount of a PDGF receptor tyrosine kinase inhibitor from any catheter-based device, intraluminal medical device or adventitial medical device comprising nanoparticles according to claim 1.

21. A method for preventing or treating restenosis comprising the controlled delivery of a therapeutically effective amount of a PDGF receptor tyrosine kinase inhibitor from any catheter-based device, intraluminal medical device or adventitial medical device comprising nanoparticles according to claim 1.

22. A method for the stabilization or repair of arterial or venous aneurisms in a subject comprising the controlled delivery of a therapeutically effective amount of a PDGF receptor tyrosine kinase inhibitor from any catheter-based device, intraluminal medical device or adventitial medical device comprising nanoparticles according to claim 1.

23. A method for the prevention or treatment of anastomic hyperplasia in a subject comprising the controlled delivery of a therapeutically effective amount of a PDGF receptor tyrosine kinase inhibitor from any catheter-based device, intraluminal medical device or adventitial medical device comprising nanoparticles according to claim 1.

24. A method for the prevention or treatment of arterial, e.g. aortic, bypass anastomosis in a subject comprising the controlled delivery of a therapeutically effective amount of a PDGF receptor tyrosine kinase inhibitor from any catheter-based device, intraluminal medical device or adventitial medical device comprising nanoparticles according to claim 1.
